(19) United States
(12) Patent Application Publication
(10) Pub. No.: US 2005/0203502 A1
(43) Pub. Date: Sep. 15, 2005

(54) Method and system for monitoring atrial fibrillation ablations with an ablation interface device

(76) Inventors: Birinder R. Boveja, Milwaukee, WI (US); Angely Widhany, Milwaukee, WI (US)

Correspondence Address: BIRINDER R. BOVEJA & ANGELY WIDHANY, P. O. BOX 210095, MILWAUKEE, WI, 53221, US

(21) Appl. No.: 11/146,460

(22) Filed: Jun. 7, 2005

Related U.S. Application Data
(63) Continuation-in-part of application No. 11/112,648, filed on Apr. 22, 2005.

(51) Int. Cl. A61B 18/18

(52) U.S. Cl. 606/32; 606/34

(57) ABSTRACT

A computer based ablation interface system provides useful information for monitoring catheter ablations, especially left sided ablations for atrial fibrillation. Such a system provides added safety, by providing useful information during the ablation procedure. The system comprises a computer, specialized software, analog and digital input/output board, transducer, and display. The information displayed includes both electrical parameters and physiological parameters. In one aspect of the invention alarms and indicators are displayed on the screen for highlighting certain events to the physicians, for guiding them through the ablation procedure. In another aspect of the invention, the system comprises circuitry for automatically switching the ablation circuit off, based on pre-determined events.

This application is a continuation of application Ser. No. 11/112,648 entitled “Method and system of increasing safety of cardiac ablation procedures” and is related to a co-pending application entitled “Method and system of stopping energy delivery of an ablation procedure with a computer based device for increasing safety of ablation procedures”. These applications are incorporated herein in their entirety by reference.

### FIELD OF INVENTION

The present invention relates to catheter ablation, more specifically system and method for monitoring catheter ablations for atrial fibrillation.

### BACKGROUND

Many cardiac arrhythmia's that formerly required the use of potentially toxic drugs or cardiac surgery can now be routinely cured (or at least palliated) in the electrophysiology laboratory by means of transcatheter ablation techniques.

As shown in conjunction with FIG. 1, the basic idea behind transcatheter ablation is to position an electrode catheter to a critical area within the heart 52, and to apply damaging energy through the catheter in order to create a discrete scar. Strategically placed scar tissue, since it is electrically inert, can disrupt the pathways necessary for pathologic tachyarrhythmias.

A novel personnel computer (PC) based system and method disclosed here, interfaces with both the patient and the ablation generator for monitoring and displaying useful information which adds safety to the ablation procedures. This invention finds uses in different types of cardiac ablation procedures, particularly left atrial ablations for atrial fibrillation, and left ventricular ablations for ventricular tachycardias (VT's).

### Background of Atrial Fibrillation (AF)

Atrial fibrillation (AF) can be paroxysmal (usually associated with normal hearts) or chronic (usually associated with heart disease). Although, atrial fibrillation is thought to be a reentrant arrhythmia of the atrial myocardium, it is not a “typical” reentrant arrhythmia. Atrial fibrillation can be induced by pacing, but unlike typical reentrant arrhythmia's, it cannot be terminated by pacing. Its continuous and chaotic atrial activity also precludes meaningful analysis by recording intraatrial electrograms. The development of AF is dependent on a complex interaction of several mechanisms that are broadly categorized as the triggers and substrate, emphasizing the importance of the management of triggers even when modifying the substrate for AF.

Atrial fibrillation is caused by multiple reentrant wavefronts, moving haphazardly across the surface of the atria like a family of tornadoes—continuously colliding with each other, extinguishing each other, and spinning off new twisters. Just as tornadoes are frequent on the uninterrupted Great Plains but almost unheard of in the choppy terrain of the Rocky Mountains, atrial fibrillation occurs only when the atria present a large surface area of contiguous, electrically homogeneous tissue. This is why small animal hearts cannot be made to fibrillate. And this is why procedures that divide the large, contiguous atrial surface into smaller regions of conducting tissue can eliminate atrial fibrillation.

It is recognized that for atrial fibrillation to sustain itself, a relatively large amount of contiguous myocardial surface must be present. Careful mapping studies of the wavefronts of atrial (and ventricular) fibrillation have shown that these arrhythmias do not use fixed reentrant circuits. Unless a critical amount of continuous myocardial surface is present, these arrhythmias would quickly extinguish themselves.

This principle also formed the basis for the original Maze procedure, a surgical technique that was developed as a cure for atrial fibrillation. As with many cardiac arrhythmias, surgical cure has been the forerunner for the development of catheter-based modification of the substrate for AF. Based on the multiple wavelet hypotheses for AF, Cox and colleagues developed a biatrial surgical procedure (the Maze operation) that segmented the atria so that reentrant activity was no longer possible. The Maze procedure requires creating a series of linear scars on the atrial surface to produce a mazelike pattern. As a result, there is simply not enough contiguous surface area for the atrial fibrillation to propagate itself. The surgical Maze procedure appears to be an effective means of ameliorating atrial fibrillation, but the surgical procedure has several significant drawbacks: significant open heart surgery is required, the atria are rendered dysfunctional in many patients, and a substantial proportion of patients still require antiarrhythmic drug therapy to maintain sinus rhythm.

Nonetheless, the fact that the Maze procedure can work validated the principle that atrial fribrillation requires a critical mass of contiguous myocardium to propagate itself. Investigators started assessing the possibility of creating linear lesions in the atria via catheter to achieve the same effect seen with the Maze surgical procedure. It had stimulated the effort to develop catheter-based “maze” procedures to treat atrial fibrillation. Transcatheter techniques have now become available to help manage this most vexing of cardiac arrhythmias. As it turns out, seven discrete linear scars (three in the right atrium and four in the left atrium) can approximate the lesions created in the surgical maze procedure.

Early catheter based procedures had been exceedingly tedious and lengthy, and had resulted in significant morbidity (the chief problem being systemic embolization secondary to having catheters in the left heart for extended periods of time). Yet these early procedures have had the major benefit of proving the potential of such a technique. A substantial proportion of the patients who have had catheter maze procedures to date (all of whom had refractory arrhythmias) were indeed rendered free of atrial fibrillation.

Because atrial fibrillation is the most common symptomatic arrhythmia, and because the present-day treatments for this arrhythmia are so deeply unsatisfying, the promise of a catheter-based technique that can “cure” atrial fibrillation is extremely attractive.

Catheter based procedures have now evolved, which predominantly involve the left atrium (LA), demonstrating a high success of restoring sinus rhythm (80% to 99% long-term cure of AF with antiarrythmic agents). This had fueled interest in the development of catheter-based techniques to modify the substrate for AF and provide hope for a similar long-term cure of AF without the need for an open-chest procedure. Technologic advancements and technical refinements have been such that catheter-based linear ablations can now be viewed as a “closed heart surgical procedure”. It seems likely that during the next decade, catheter ablation of atrial fibrillation would become a commonly performed electrophysiologic procedure.

Because these procedures are performed on the left side of the heart, where embolization is a serious concern, monitoring for both physiological parameters, and electrical parameters during the procedure is crucial. This patent application is directed to novel method and system for monitoring the ablation procedure, and under certain conditions either shutting off power to the ablation generator or disconnecting the ablation circuit.

### PRIOR ART

Prior art search reveals U.S. patent Application publication No. 2004/0,078,036 A1 (Keidar) is generally directed to real-time monitoring and mapping of ablation lesion formation in the heart. The means, functionality, and intent of the Keidar '036 application is significantly different than applicant's disclosure. In the Keidar '036 disclosure a conventional mapping system is used with an endocardial mapping and ablation catheter, to monitor energy delivery to individual lesions and represent the lesion pictorially on a computer generated map. Among other things, the intent is “if user 22 determines that a particular site or region has not been sufficiently ablated, he can immediately return to the site and repeat the ablation procedure” paragraph [0133], lines 20-22, on page 8.

In contrast, in applicant's system and method, the patient is monitored, as well as, the whole ablation procedure is monitored as opposed to individual lesions, using an ablation interface system, where both electrical parameters and physiological parameters are measured, processed, and displayed. Further, in applicant's disclosure if predetermined conditions are reached, a software guided instrument based interruption of energy delivery occurs which acts as safety alarm, and which may be simply reset to continue on with the procedure.

Even though the atrial fibrillation ablation procedures are evolving, and technology supporting these procedures is also evolving, in the current art, the monitoring for the whole cardiac procedure (as opposed to individual lesion) is limited. Currently during these procedures, there is no indication of how much cumulative energy is used in the procedure. Yet, electrical and physiological information about the whole procedure is important for ablation procedures on the left side of the heart, where there is increased risk of thrombus formation and emboli breaking off, possibly leading to serious complication for the patient.

### SUMMARY OF THE INVENTION

The current invention is directed to novel method and system for monitoring the ablation procedure, and under certain conditions shutting-off energy delivery to the tissues.

Accordingly, it is an object of the invention to monitor an index of the whole ablation procedure via an ablation interface device (AID), by obtaining electrical signals and data non-invasively into a computer via an input/output (I/O) interface device.

It is another object of the invention, to obtain physiological data into ablation interface device, via a keyboard.

It is another object of the invention, to obtain information from electrical signals and physiological data that is useful and critical to increase safety of the ablation procedure.

It is another object of the invention, to process and display the information for use in monitoring the procedure.

It is another object of the invention, that alarms are triggered based upon reaching predetermined threshold values.

It is another object of the invention, to use the ablation interface device (AID) for left-atrial procedures, such as ablation for atrial fibrillation.

It is another object of the invention, to use the ablation interface device (AID) for ablation procedures in the left ventricle.

It is another object of the invention, to shut-off ablation energy based on meeting predetermined threshold values.

It is another object of the invention, to have control switching means inserted in the ground loop of the ablation circuit.

It is another object of the invention, to have control switching means inserted on the catheter side of the ablation circuit.

It is another object of the invention, for the control switching means to comprise a relay switch and controlling circuitry for said relay switch.

In one aspect of the invention, non-invasive electrical signals can be obtained from an ablation generator.

In another aspect of the invention, the software used is one from a group comprising, Lab Windows/CVI, LabView (National Instruments Corp.), Microsoft Visual C++, Dot Net framework, MATLAB, Microsoft Visual Basic, or other functionally equivalent language.

In another aspect of the invention, useful electrical information such as cumulative energy delivered, as well as, physiological information such as active clotting time (ACT), and other useful information are displayed.

In another aspect of the invention, electrical signals obtained from an ablation generator are incorporated into the ablation interface device (AID).

In another aspect of the invention, information from a mapping system may be incorporated into the ablation interface device (AID).

In yet another aspect of the invention, fluoroscopy image may be displayed along with information from the ablation interface device (AID).

Various other features, objects and advantages of the invention will be made apparent from the following description taken together with the drawings.

### BRIEF DESCRIPTION OF THE DRAWINGS

For the purpose of illustrating the invention, there are shown in accompanying drawing forms which are presently preferred, it being understood that the invention is not intended to be limited to the precise arrangement and instrumentalities shown.

FIG. 1 depicts general concept for cardiac ablation procedures.

FIG. 2 depicts a general setup of cardiac ablation procedures.

FIGS. 3A and 3B are two views of left atrium showing numerous (approximately over 100) ablation tags, in electroanatomical map from a commercially available mapping system.

FIG. 4 is a simplified block diagram depicting radio-frequency (RF) ablation circuit and ablation interface device (AID) of the current invention.

FIG. 5 is a schematic diagram of one form of transducer and signal conditioning unit used to get signals into the ablation interface device (AID).

FIG. 6A is a simplified block diagram of one configuration, where signals from ablation generator and other signals are incorporated into the ablation interface device.

FIG. 6B depicts display from the configuration shown in FIG. 6A.

FIG. 7A is a simplified block diagram of one configuration, where signals from a mapping system are incorporated into the ablation interface device.

FIG. 7B depicts display from the configuration shown in FIG. 7A.

FIG. 8A is a simplified block diagram where computer controlled stopping of ablation can occur via a control switch in the ground loop.

FIG. 8B is a simplified block diagram where computer controlled stopping of ablation can occur via a control switch on the catheter side of the ablation circuit.

FIG. 9 is one configuration of ablation interface unit used in conjunction with fluoroscopic imaging.

FIG. 10 is one configuration of ablation interface unit used in conjunction with a mapping system.

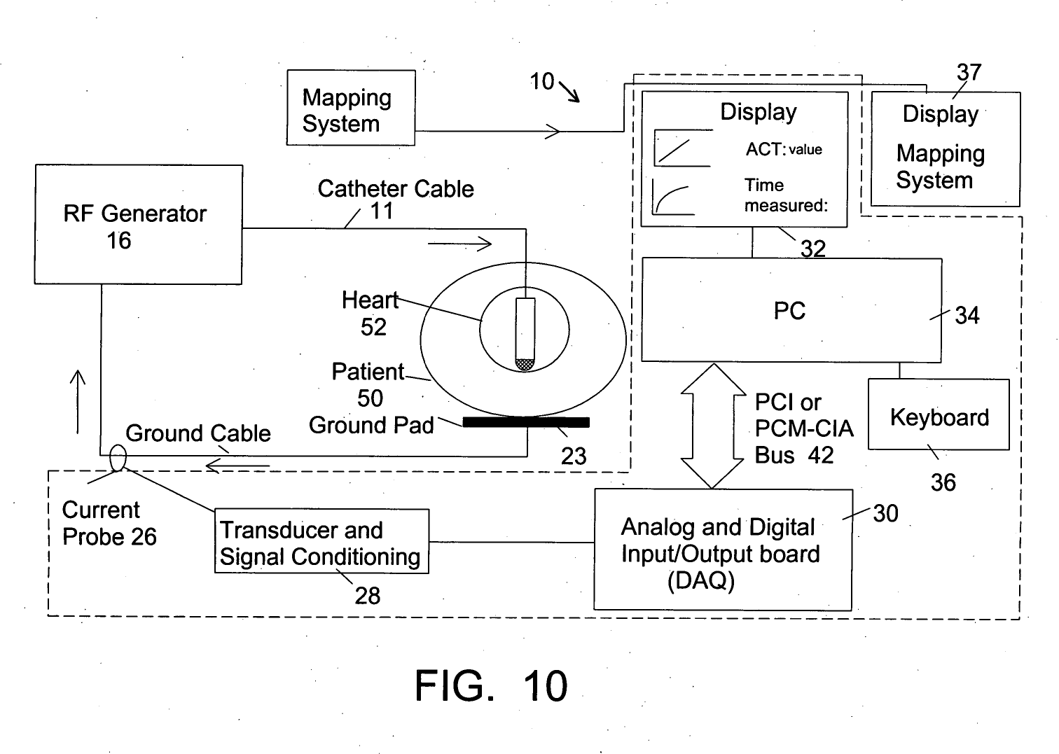

FIG. 11 is one configuration of ablation interface unit used in conjunction with a mapping system and fluoroscopic imaging.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

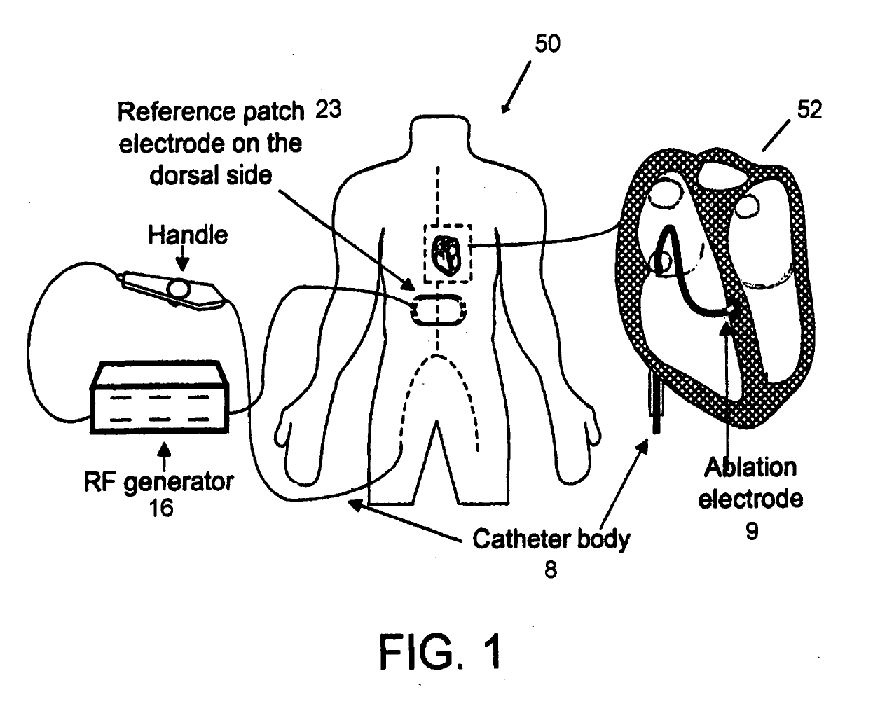

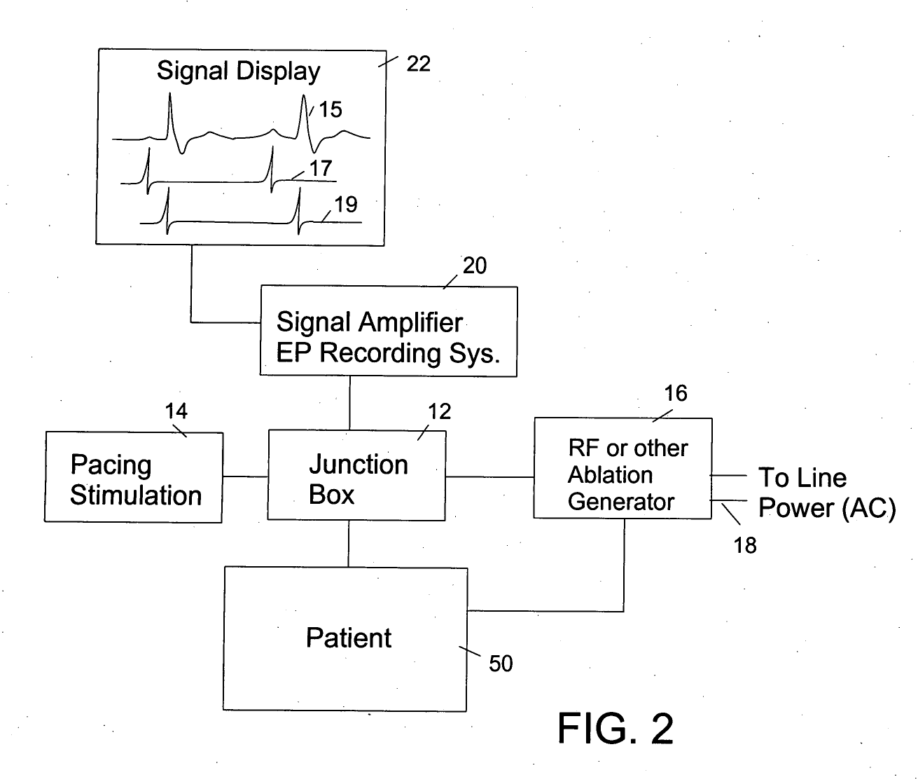

For the purposes of explaining the methodology of the current invention, it is instructive to understand a typical setup for a generic cardiac ablation procedure. Shown in conjunction with FIG. 2 is a typical setup, where body surface ECG 15 (usually 12 lead), atrial intra-cardiac (IC) signal 17, ventricular intra-cardiac (IC) signal 19, and other intra-cardiac (IC) signals such as His bundle recording (not shown) and coronary sinus signals (not shown) are obtained from the patient 50, via transvenous diagnostic catheters. The catheters in the body are connected via extension cables to a junction box 12. The body surface signals are typically amplified by amplifiers of an EP recording system 20 and displayed on a display monitor 22 for easy visualization during the Electrophysiology (EP) study and cardiac ablation procedure. A pacing stimulator 14 is also connected (typically via junction box 12) for pacing of different sites within the heart such as the atrium or ventricle for example. An ablation generator 16 is connected to the patient 50. If a radiofrequency (RF) generator is used, a ground patch 23 or reference patch 23 which is typically connected on patient's back (FIG. 1) is connected to the RF ablation generator, and an ablation catheter 37 positioned inside the heart 52 and connected to the RF ablation generator 16 via connector cables completes the circuit for ablation procedure to proceed.

Using the above connection methodology, radiofrequency ablations are routinely performed for a variety of cardiac arrhythmia's. One of the fastest growing procedures, and one of the most challenging ablations is for the treatment of atrial fibrillation (AF). Emerging evidence has suggested that the substrate for AF may relate to diffuse atrial structural remodeling and its electrophysiologic consequences. Catheter ablation to modify the substrate is becoming a popular procedure.

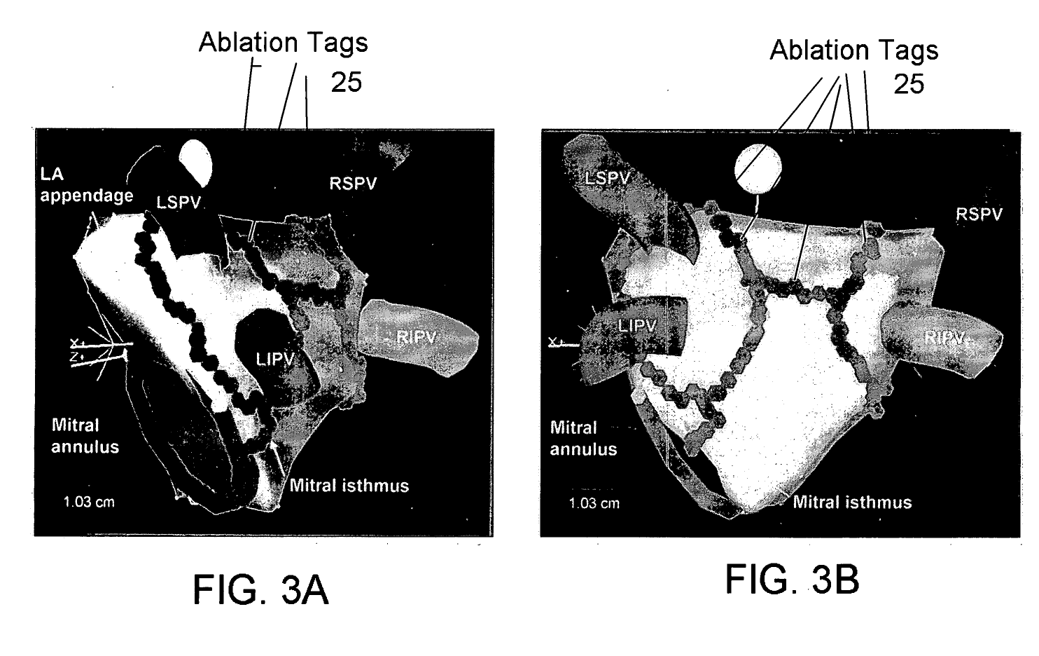

FIGS. 3A and 3B show two different views of a left atrium using an electroanatomic map with a mapping system. Shown on these left atrial geometry maps are numerous ablation tags (approximately over 100). Each ablation tag represents a physical spot where an ablation was performed, usually for 10-60 seconds. Because these ablation energies are applied on the left side of heart, there is an increased risk of thrombus formation, and an emboli breaking off on the arterial side of the circulation. An important parameter to track is the cumulative energy delivered during the ablation procedure, which is currently not being tracked.

Another important parameter to track is the coagulation status of the patient. Usually after transeptal puncture a Heprin IV bolus (approximately 100 U/Kg) is given. Thereafter, a heprin drip is maintained to keep the activated clotting time (ACT) to about 300 seconds (between 275 and 350 seconds). The second precaution that is taken is to minimize the total amount of ablation performed in the left side of the heart.

The method and system of this invention aids in the convenient monitoring of vital parameters such as cumulative energy delivered to the left atrium, ACT level of the patient, as well as, other optional useful parameters during the left atrial ablation procedure for atrial fibrillation. In one aspect the appropriate parameters are displayed along with indicators and alarms. In this embodiment the physician monitors the physiological and electrical parameters, along with visual alarms on the display screen. The physician uses his/her medical knowledge to decide what steps, if any, should be taken based on the indicators and alarms. At the end of the procedure, a summery report can be printed for medical record keeping.

In another aspect, the software and hardware may be configured such that, based on certain pre-determined parameter thresholds the software may switch the ablation generator off. This would give the physician a chance to reposition the catheter, before proceeding further with the procedure.

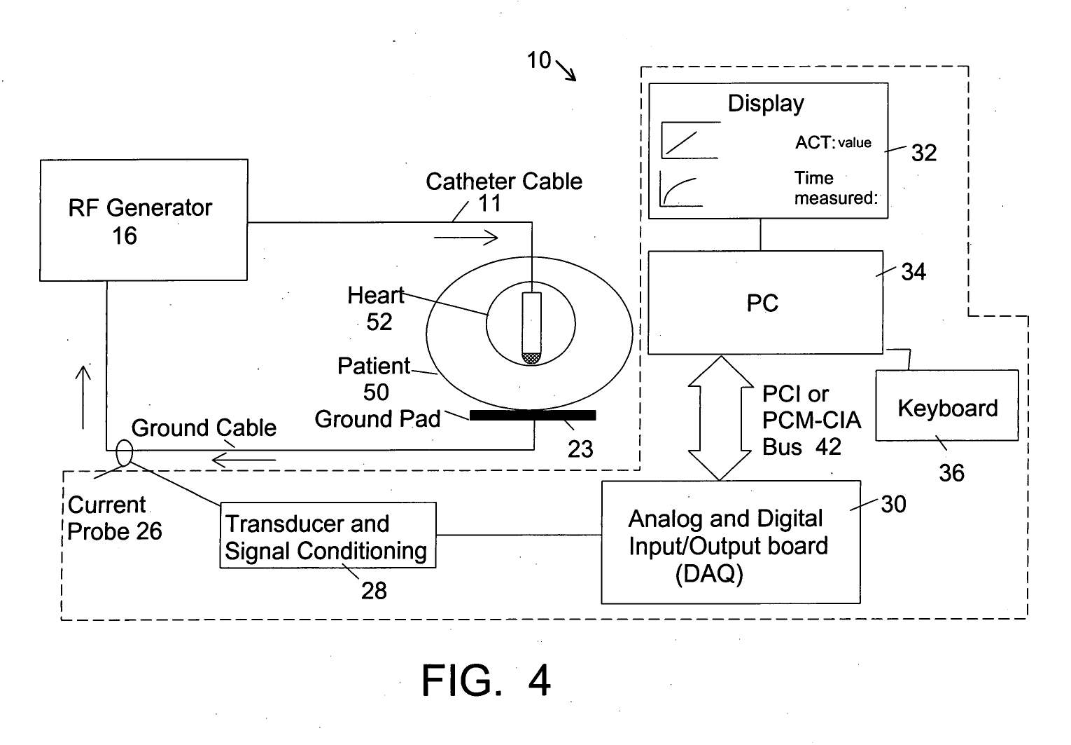

FIG. 4 is a simplified block diagram of the Ablation Interface Device (AID) 10 of the invention. A current probe 26 non-invasively placed on the ground cable is used for measuring current and deriving total current, total energy measurements which are displayed on the display unit 32. Alternatively, the current probe may be placed on the catheter side of the ablation circuit. The display unit 32, displays both instrument measured and derived parameters, as well as, keyboard entered information, such as physiological parameters. Advantageously, the combination of the collective information displayed, provides useful information about the ongoing procedure. Additionally, the software is also configured to display alarms and triggers. For example, when the cumulative energy delivered reaches a predetermined safety limit level, an indicator displayed on the display 32 screen indicates this. Therefore, at any time during the procedure, the physician would know via the graphs and indicators, how far (or close) they are to the recommended safety limit, among other things.

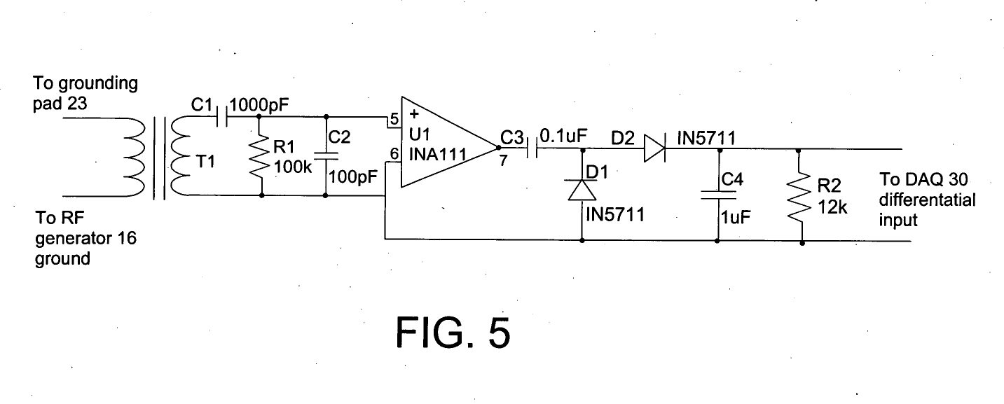

Standard current probes 26 such as available from Tectronix Corp. may be used. Signal conditioning block is shown in conjunction with FIG. 5. T1 is torroidal transformer, with input one turn and output 10 turns. It is also possible to use a shunt resistor (one Ohm or less) in place of the transformer. The shunt resistor is inserted in series with the ground lead. In this FIG. U1 is set to gain of ten. D1 and D2 are small signal Schottky diodes. The diode converts the approximately 500 KHz AC signal of RF ablation to a steady DC voltage, and R2 sets the decay on the output. It will be clear to one skilled in the art, that other functionally equivalent circuitry may be used.

Shown in conjunction with FIG. 4, the signals from Transducer and Signal Conditioning Unit 28 are connected to data acquisition board (DAQ) board 30. Other interface devices such as GPIB may also be used. Other analog and digital signals may also be obtained simultaneously.

The computer 34 is a personnel computer in the presently preferred embodiment, which may be either a desktop or a sufficiently powerful laptop computer. The computer is configured with the appropriate interface drivers and appropriate software for manipulating and analyzing data. The presently preferred embodiment utilizes LabView software (available from National Instruments, Austin, Tex.). It will be clear to one skilled in the art, that other software such as Lab Windows/CVI, Microsoft Visual C++, Dot Net framework, MATLAB, and Microsoft Visual Basic, among others may be used. Use of any of these or other comparable languages for this purpose that are available now or developed in the future, is considered within the scope of the invention.

As was previously mentioned, since ablation for atrial fibrillation is performed in the left atrium, physicians are careful about limiting the total amount of ablation energy delivered. Accordingly, the display 32 will not only show the parameters (Impedance, tip temperature, time, energy) for the current ablation, but also cumulative values. Additionally, other pertinent information entered via the keyboard, such as ACT and time it was measured will also be conveniently displayed. Other physical or physiological information e.g. arterial blood pressure will also be displayed at one place. Advantageously, by having the relevant information displayed in a meaningful way, adds safety and provides convenience to the ablation procedure.

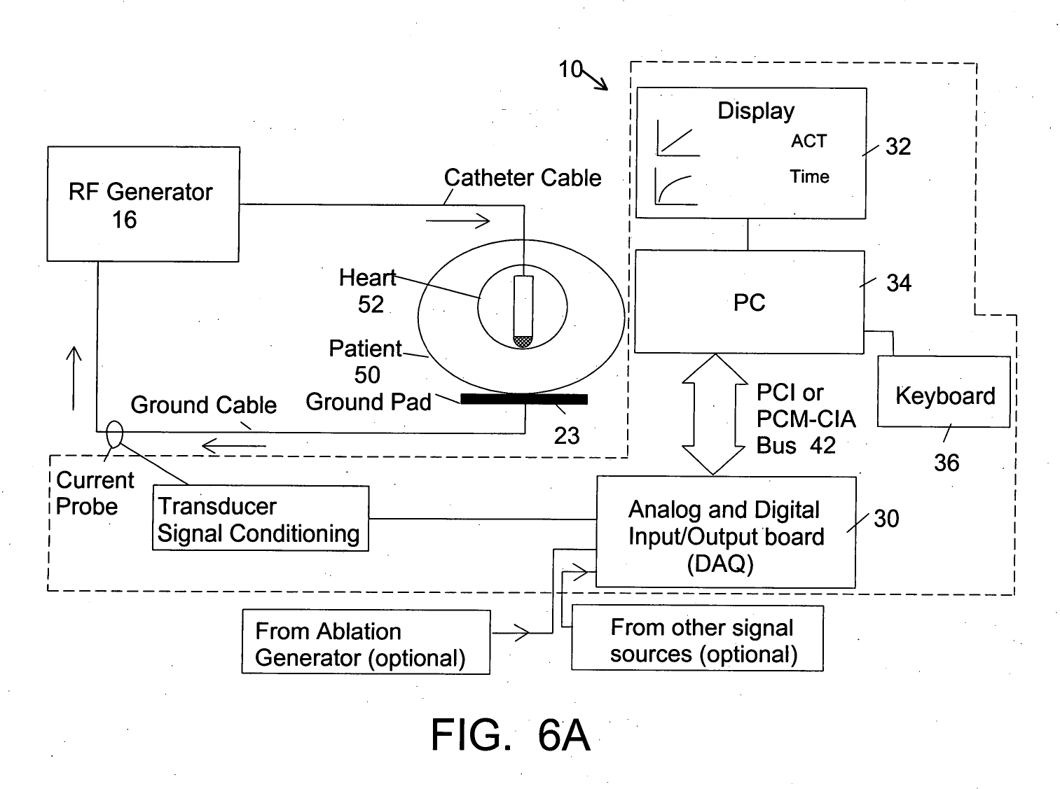

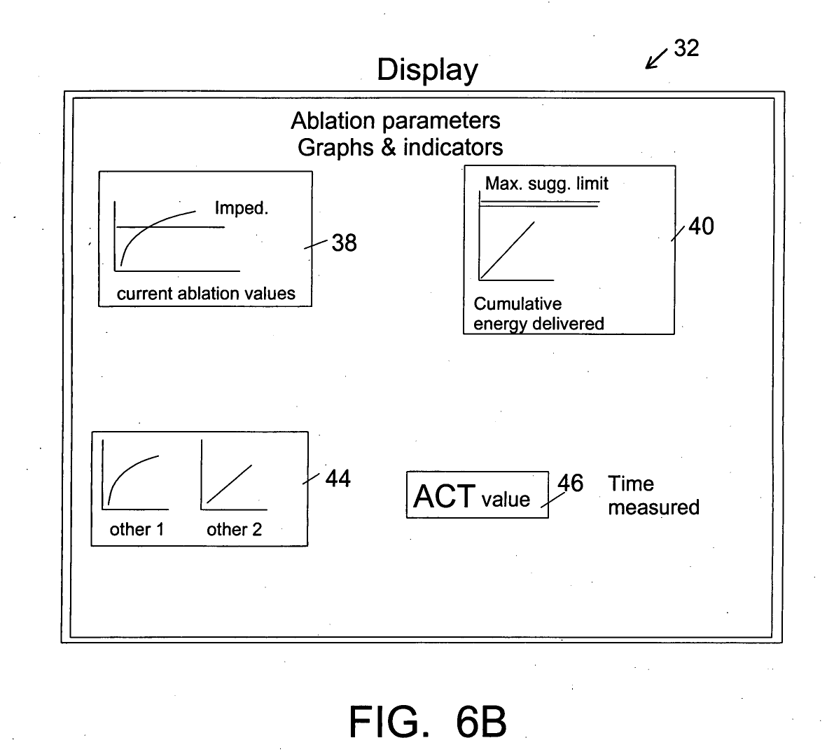

Shown in conjunction with FIG. 6A, signals from ablation generator 16 may also be incorporated into the ablation interface device (AID) 10, via the multi-channel input of data acquisition board 30. During the time that the ablation is ON, the signals for providing information on the current ablation is displayed on the display screen of the AID 10. This is shown in conjunction with FIG. 6B. The current ablation graph 38 is displayed in addition to cumulative energy graph 40, along with other information.

The information in this embodiment is manipulated and processed using LabView Software, LabView is a graphical programming environment as opposed to line coding. Other software mentioned previously would work equally as well.

One example of display (without limitation) is shown in conjunction with FIG. 6B. The software is configured, such that in addition to the parameters of the current ablation, the cumulative energy and other parameters are also displayed.

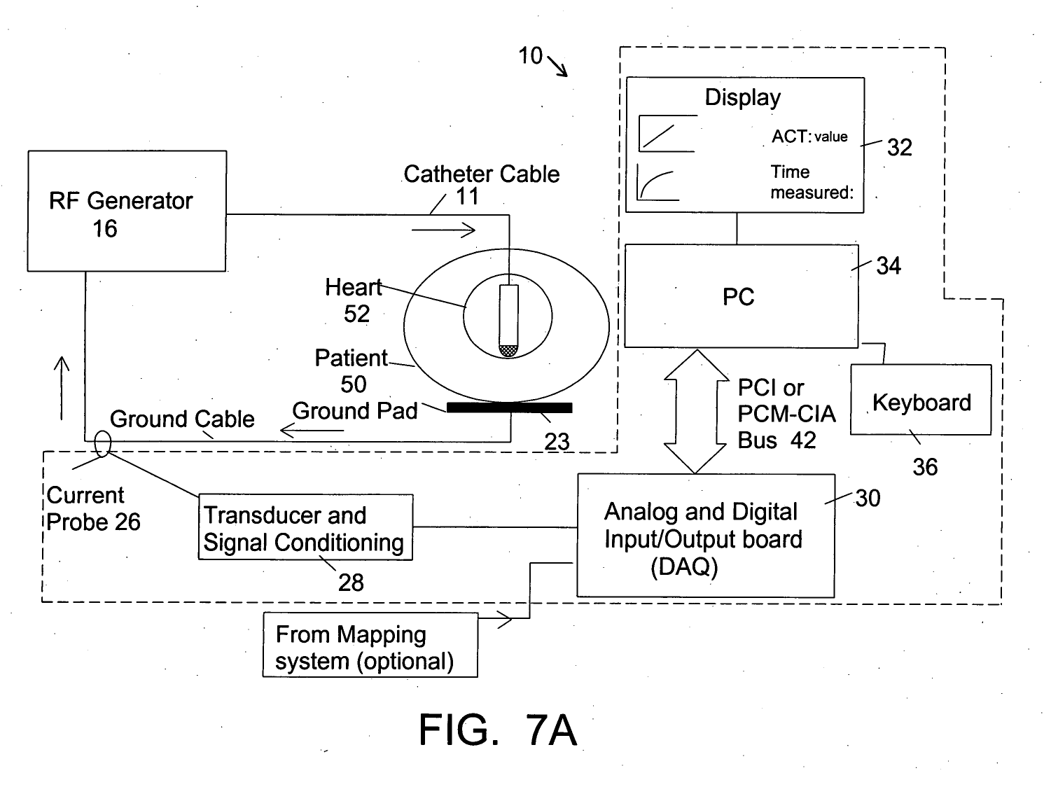

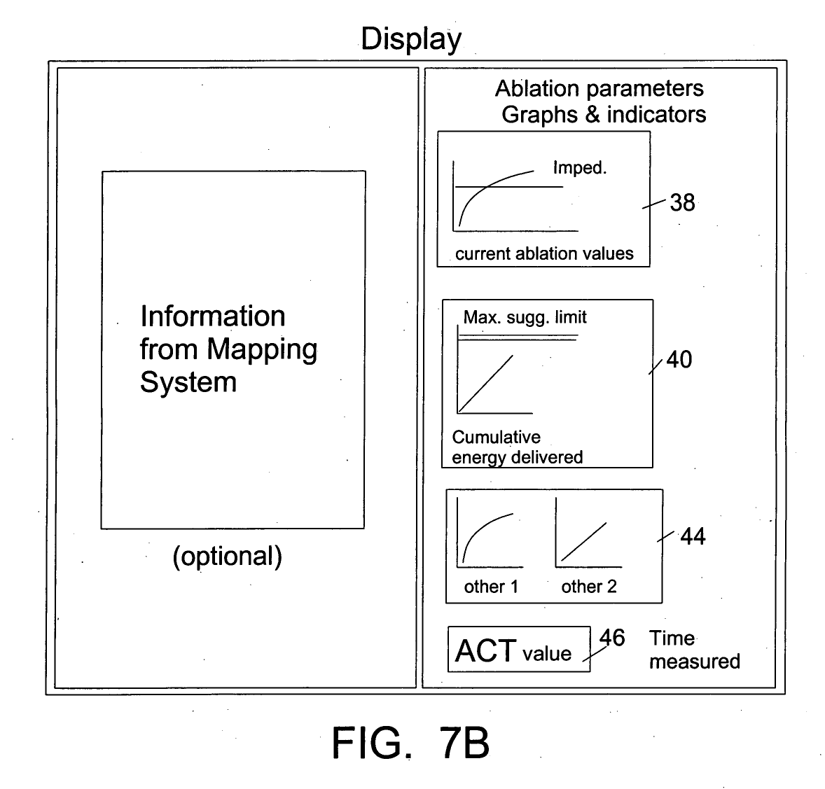

As shown in conjunction with FIG. 7A, in one aspect, information signals from a mapping system may also be incorporated into an ablation interface device (AID) 10. In this embodiment, the signals from mapping system are incorporated via the multiple channels of data acquisition board (DAQ) 30. This may be in addition to signals from ablation generator as was shown in FIG. 6A. An example of display for this embodiment (without limitation) is shown in conjunction with FIG. 7B. In this embodiment, the mapping system display is present adjacent to the other information. The other information is same as described in conjunction with FIG. 6B The mapping system may be any of the commercially available cardiac mapping system. Examples of four cardiac mapping systems are:
a) Carto mapping system, which is an electroanatomical mapping system available from Biosense/Webster (Diamond Barr, Calif.);
b) Navix or Ensite mapping systems available from Endocardial Solutions, a division of St. Jude Medical (MN);
c) RPM mapping system available from Boston Scientific; and
d) Local Lisa mapping system available from Medtronic Inc. (Minneapolis, Minn.).

Other mapping systems, yet to be developed may similarly be interfaced with the current invention.

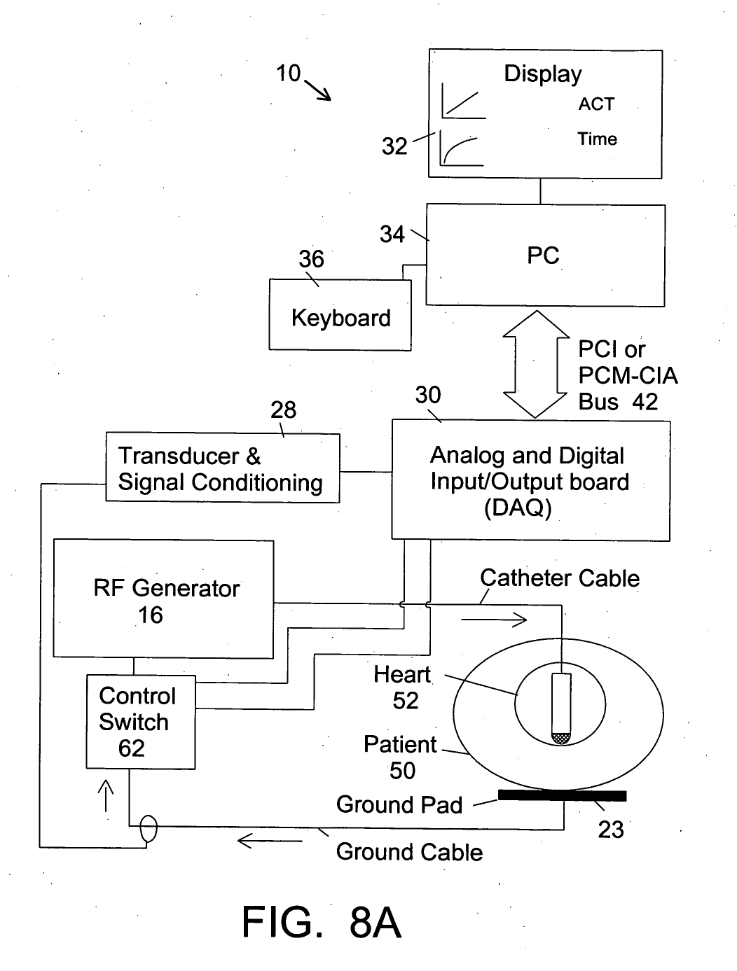

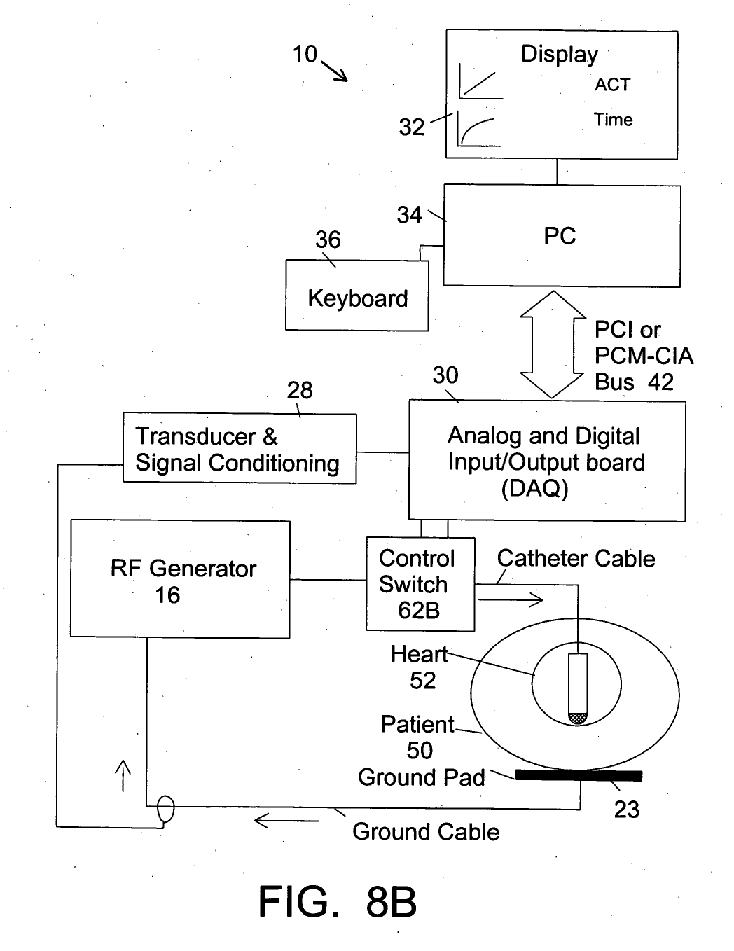

In one aspect of the invention, shown in conjunction with FIGS. 8A and 8B, if some aspect of safety limit is exceeded based on pre-determined levels, the software automatically trips the ablation circuit open, whereby disrupting the energy delivery to the cardiac tissue. A control mechanism for automatically shutting off ablation is disclosed in a co-pending application. As shown in conjunction with FIG. 8A, in one preferred embodiment, the control switch 62A mechanism is placed on the ground loop connection of the ablation circuit. Alternatively, as shown in conjunction with FIG. 8B, the control switch may also be placed on the catheter side of the ablation circuit. Examples of safety limits, without limitation, may be the total amount of energy delivered, total amount of current delivered, sudden change in impedance beyond a predetermined limit. Other parameters may also be used based on physician preference.

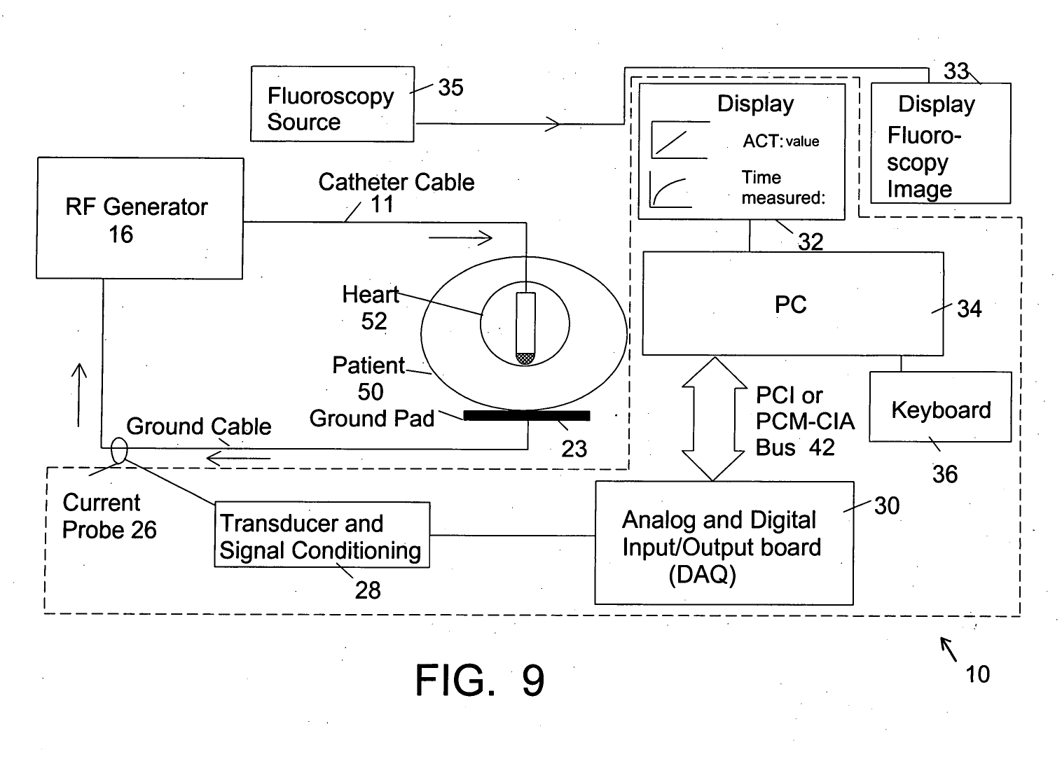

Shown in conjunction with FIG. 9, is another configuration for using the ablation interface device (AID) 10 of the current invention. In this configuration, the fluoroscopy image (video) signals go directly to a display 33 device (monitor), which may be mounted or physically placed adjacent to the display 32 (monitor) of the AID 10. The advantage of this configuration is that a physician performing an atrial fibrillation ablation procedure has both visual or catheter location information, as well as, derived electrical parameter information, to guide the physician to safely perform the ablation procedure.

It will be clear to one skilled in the art, that as cardiac imaging systems are improved, or newer technologies are developed, they can be incorporated with the AID 10 of this invention. For example, a new imagining technology being developed would superimpose a digital image such as a CT scan or MRI scan, on the fluoroscopic image of the heart during a live procedure. Such combined image, where the coordinates of CT or MRI image of the heart are registered to the fluoroscopic live image, provide detailed information about the catheter position in the left atrium. The information provided from the AID 10 of the current invention provides detailed electrical information about the individual and cumulative ablation parameters. The two sets of information, anatomic and electrical parameter information guides the physician to a safe ablation procedure for atrial fibrillation.

Similar to the configuration in FIG. 9, the mapping system may also be connected independently of AID 10, except for the displays from mapping system and AID 10 are mounted or physically placed adjacent to each other. This combination will also provide useful information to the physician during the ablation procedure.

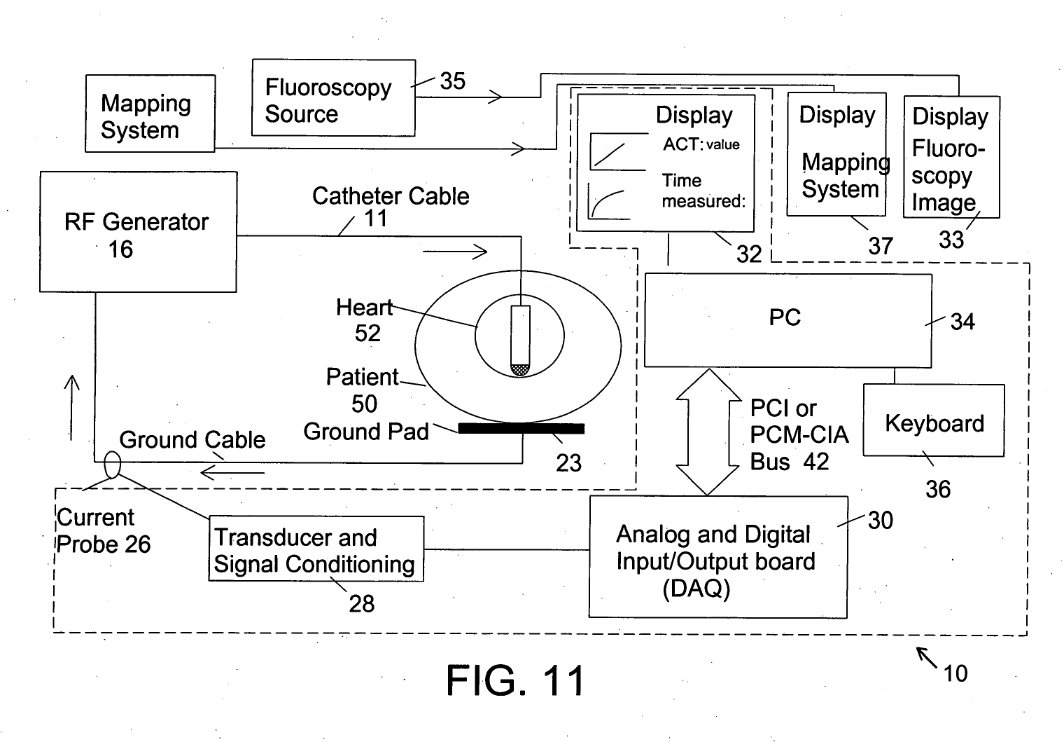

In yet another configuration, as shown in conjunction with FIG. 11, both fluroscopy and mapping system are connected independently, except that the displays of AID 10, mapping system, and fluoroscopic image is mounted or placed adjacent to each other. As before, the combined information is displayed for the physician in a useful manner, and is a powerful tool for guiding the physician through a safe ablation procedure.

Even though this invention has been disclosed for use with left atrial ablations it applies equally well to left ventricle. Left ventricular ablations are frequently performed for ventricular tachycardias (VT) in ischemic hearts. The rationale for using AID 10 is the same as using it for left atrial ablations. In both cases, a large number of ablation may be performed on the left (arterial) side. Again, the total amount of energy needs to be monitored, along with physiological parameters such as patient's ACT.

The present invention may be embodied in other specific forms without departing from the spirit or essential attributes thereof. It is therefore desired that the present embodiment be considered in all aspects as illustrative and not restrictive, reference being made to the appended claims rather than to the foregoing description to indicate the scope of the invention.

1. A method of monitoring a cardiac atrial fibrillation ablation procedure, comprising the steps of: obtaining non-invasively electrical signals and/or physiological data into a computer, processing said signals and said data into useful information about said ablation procedure, and displaying said information on a monitor means to view said information and/or to initiate a manual or automatic shut-off of energy delivery of said procedure, when a predetermined event occurs.

2. A method of monitoring cardiac atrial fibrillation ablation procedure, comprising the steps of:
obtaining non-invasively electrical signals into a computer means via an analog and digital, input and output (I/O) interface means, and obtaining physiological data via a keyboard means;
processing said signals and said data by said computer using a software/program means into useful information and measuring against at least one predetermined threshold;
displaying said information on a monitor;
detecting a predetermined event based on said at least one threshold level; and
taking action by triggering a visual alarm or initiating an automatic shut-off of energy delivery of said procedure via a control switching means, or as decided by the physician.

3. The method of claim 2, wherein said non-invasive electrical signals can also be obtained from an ablation generator.

4. The method of claim 2, wherein said software means for processing and measuring said signals can be coded using one from a group comprising, Lab Windows/CVI, LabView (National Instruments Corp.), Microsoft Visual C++, Dot Net framework, Microsoft Visual Basic, or functionally equivalent software.

5. The method of claim 2, wherein said information about said procedure comprises ablation parameters, graphs and indicators, impedance, cumulative energy delivered, maximum suggested limit, activated clotting time (ACT) value at different time periods, and any other indicator about the ablation procedure obtained from said signal and said data.

6. The method of claim 2, wherein said control switching means is inserted in the ground loop of the ablation circuit.

7. The method of claim 2, wherein said control switching means is inserted in the catheter side of the ablation circuit.

8. The method of claim 2, wherein said control switching means comprises a relay switch and controlling circuitry for said relay switch.

9. The method of claim 2, wherein said information is further used to develop safe standards for cumulative energy delivery.

10. The method of claim 2, wherein mapping and/or fluoroscopy imaging information can also be displayed with said information for viewing together.

11. The method of claim 2, wherein said monitoring for said cardiac procedures may be in the left-ventricle.

12. A system for monitoring cardiac atrial fibrillation ablation procedure, comprising:
an analog and digital, input and output (I/O) interface means for obtaining electrical signals, and a keyboard for obtaining physiological data into a computer;
said computer and a software means to process said electrical signals and/or physiological ablation data into useful ablation information, and comparing said signals and said data with at least one predetermined threshold level to detect a predetermined event;
monitor means for viewing said ablation information; and
a control switch means for automatic shut-off of said ablation procedure by said computer or shut-off as decided by a physician.

13. The system of claim 14, wherein said non-invasive electrical signals can be obtained signal probe means or from an ablation generator.

14. The system of claim 14, wherein said an analog and digital interface means is further connected to a transducer and signal conditioning means.

15. The system of claim 14, wherein said software means for measuring and processing said signals can be written using one from a group comprising, Lab Windows/CVI, LabView (National Instruments Corp.), Microsoft Visual C++, Dot Net framework, Microsoft Visual Basic.

16. The system of claim 14, wherein said information about said procedure comprises ablation parameters, graphs, and indicators, cumulative energy delivered, maximum suggested limit, activated clotting time (ACT) value at different time period, and any other indicator about the ablation procedure obtained from said signal and said data.

17. The system of claim 14, wherein said control switching means is inserted in the ground loop of the ablation circuit.

18. The system of claim 14, wherein said control switching means is inserted in the catheter side of the ablation circuit.

19. The system of claim 14, wherein said control switching means comprises a relay switch and controlling circuitry for said relay switch.

20. The system of claim 14, wherein output from mapping systems and/or fluoroscopy imaging information can also be displayed with said information.
